SEQUENCE LISTING (1 sequence)

SEQ ID NO: 1 - protein, 12 aa, Plasmodium vivax
EEELEATPEDDF

(19) United States
(12) Patent Application Publication
(10) Pub. No.: US 2002/0102620 A1
(43) Pub. Date: Aug. 1, 2002

(54) ANTIBODIES AND PEPTIDES FOR DETECTION OF PLASMODIUM VIVAX

(76) Inventors: S. MELISSA MARET, YARDLEY, PA (US); RICHARD L. MALLONEE, BALTIMORE, MD (US); DAVID W. BAGGETT, STEWARTSTOWN, PA (US); MICHELLE C. CARRIGAN, SHREWSBURY, PA (US)

Correspondence Address: BECTON DICKINSON AND COMPANY, 1 BECTON DRIVE, FRANKLIN LAKES, NJ, 07417

(21) Appl. No.: 09/306,905

(22) Filed: May 7, 1999

(51) Int. Cl. G01N 33/53

(52) U.S. Cl. 435/7.22

(57) ABSTRACT

Novel peptides comprising an amino acid sequence which is repeated in the P. vivax ESP-1 protein (PvESP-1) are disclosed. Also disclosed are antibodies generated in response to immunization with these peptides which exhibit high specificity and sensitivity for P. vivax antigens in diagnostic assays. Assay methods employing the inventive antibodies and kits for carrying out the inventive methods are also provided.

### FIELD OF THE INVENTION

The present invention relates to antibodies which recognize and bind to a repeated sequence in the ESP-1 protein of Plasmodium vivax, to peptides for generating the antibodies and to assays employing the antibodies.

### BACKGROUND OF THE INVENTION

Plasmodium vivax and Plasmodium falciparum are the two most common causes of human malaria. Upon initial infection, sporozoites enter the hepatocytes of the host mammal and multiply by schizogony to produce merozoites. The infected cells then rupture, releasing merozoites into the blood where they enter erythrocytes and begin an asexual reproductive phase. Malarial parasite protein antigens are found in the plasma of infected individuals during acute infections, partially due to the rupture of infected erythrocytes which allows infective merozoites to invade additional erythrocytes. Parasite antigens are also released during intraerythrocytic growth of the parasite by transport of the parasite protein across the cell membrane of the infected erythrocyte. The HRP-II protein of P. falciparum is released into the plasma in this manner and detection of HRP-II has formed the basis for specific assays for diagnosis of P. falciparum malaria (Howard, et al. 1986. J. Cell Biol. 103, 1269-1277; WO 89/01785; Knapp, et al. 1988. Behring Inst. Mitt. 82, 349-359; Wellems, et al. 1987. Cell 49, 633-642; U.S. Pat. No. 5,130,416; U.S. Pat. No. 5,478,741).

J. W. Barnwell (U.S. Pat. No. 5,532,133) identified two species-specific blood stage protein antigens in P. vivax known as P. vivax Erythrocyte Secreted Protein-1. (PvESP-1) and P. vivax Erythrocyte Secreted Protein-2 (PvESP-2). These antigens reportedly present unique P. vivax-specific epitopes, making them useful in differential determination of P. vivax merozoites. Polyclonal antibodies produced in response to immunization with isolated PvESP-1 are described. Also disclosed is monoclonal antibody mAb 1D11.G10 which recognizes the PvESP-1 protein and was produced by immunization of a mouse with P. vivax infected red blood cells. Antibodies generated in response to immunization with these either PvESP-1 or PvESP-2 may be used in assays not only for diagnosis of malaria, but also identification of P. vivax as the causative agent. In P. vivax-infected Saimiri monkeys antibodies raised to the PvESP-1 and PvESP-2 antigens detected 1000 parasites/μl of blood. In humans, early acute infections were also detected. However, there remains a need for species-specific anti-P. vivax antibodies which have a higher affinity than those previously reported, in order to increase the sensitivity and specificity of the assay.

### SUMMARY OF THE INVENTION

The present invention provides novel peptides comprising an amino acid sequence which is repeated three times in the P. vivax ESP-1 protein (PvESP-1). Antibodies generated in response to immunization with these peptides exhibit high specificity and sensitivity for P. vivax in diagnostic assays.

### DETAILED DESCRIPTION OF THE INVENTION

The immunogenic peptides of the invention were identified by amino acid sequence analysis of PvESP-1 using a computer program. In this way, it was discovered that a 12-mer sequence Glu-Glu-Glu-Leu-Glu-Ala-Thr-Pro-Glu-Asp-Asp-Phe (SEQ ID NO: 1) was repeated three times in the protein. This 12-mer peptide was selected for testing as a P. vivax-specific epitope in an attempt to generate antibodies with higher affinity for the PvESP-1 antigen than antibodies previously reported.

In a preferred embodiment, addition of an N-terminal Cys and Gly to SEQ ID NO:1 allows coupling of the peptides to maleimide derivatized carrier proteins through the SH group. When used for immunization, it is preferred that the peptides be coupled to an immunogenic carrier for immunization, as coupling allows the small antigenic determinant peptides (which are haptens) to elicit an antibody response. Commonly used immunogenic carriers useful for coupling to haptens such as the peptides of the invention are listed in Immunology, An Illustrated Outline by David Male, Gower Medical Publishing, 1986, pg. 31. Methods for coupling carriers to haptens through sulfhydryl groups are known in the art and any of these are suitable for coupling the selected carrier to the immunogenic peptides of the invention. For example, see the coupling protocols described in Current Protocols in Immunology, J. E. Coligan et al., eds., Greene Publishing Assoc. and Wiley Interscience, 1992, Chapter 9. It is also believed that the N-terminal Cys-Gly serves as a spacer linkage between the carrier and the peptide. Such a spacer linkage may reduce the negative effects of the carrier on the conformation of the peptides, thus allowing the peptides to assume a conformation more characteristic of a naturally-occurring epitope of the ESP-1 protein. This more authentic conformation, in addition to the nature of the epitope itself, may contribute to the ability of the peptides to elicit the high sensitivity antibodies of the invention.

The peptides of the invention may be chemically synthesized using any of the synthetic methods known in the art, for example the solid-phase method of Merrifield (1969. Advan. Enzymol. 32:221) or the modified solid-phase methods of Sheppard and Atherton (WO 86/03494) which are now automated. Alternatively, they may be produced by expression of a recombinant oligonucleotide coding for the 12-mer or for the 12-mer and the N-terminal Cys-Gly. Methods for synthesizing a nucleic acid sequence which codes for the desired amino acid sequence, cloning it and expressing it in a transformed host cell are well known and within the ordinary skill in the art. Optionally, a nucleic acid sequence coding for a histidine tail on the peptide and a Factor Xa cleavage site between the histidine tail and the peptide may be included in the recombinant oligonucleotide. This construct allows purification of the peptide on a nickel chelate column and release of the peptide from the histidine tail by cleavage with Factor X.

Antibodies produced in response to immunization with the peptides of the invention are highly specific for P. vivax and exhibit significantly improved sensitivity in diagnostic assays as compared to the antibodies described in the prior art. This represents an important advance in the ability of clinicians to detect the specific cause of malarial disease at an early stage. The anti-peptide antibodies may be either polyclonal or monoclonal, and are produced using any suitable method for immunizing animals as is known in the art. See Current Protocols in Immunology, supra. In general, an immunogenic amount of peptide/carrier conjugate is dissolved or suspended in a physiological buffer, e.g., phosphate buffered saline (PBS), usually mixed with an adjuvant such as complete Freunds adjuvant. Animals are initially immunized with this mixture and thereafter boosted with additional doses of peptide/carrier conjugate. The immunization with peptide/carrier conjugate is then repeated with an adjuvant such as incomplete Freunds adjuvant. At about 7 to 12 weeks after the initial immunization the serum is generally tested using methods known in the art to determine the titer of antipeptide antibodies (e.g., reactivity with the immunogen in an ELISA). Modifications and adjustments to this basic immunization protocol to obtain optimal antipeptide antibody titers for any particular peptide/carrier conjugate are within the ordinary skill in the art. If purified polyclonal antibody is desired, it may be isolated from the immune serum using well-established methods, such as separation on a peptide affinity column.

The spleen cells of an animal immunized with the immunogenic peptides may be fused with murine myeloma cells for production of monoclonal antibodies using the methods of Kohler and Milstein (1975. Nature 256:495-497) or a modification of this method as is known in the art (Oi and Herzenberg. 1980. Selected Methods in Cellular Immunology, Mishell and Shiigi, eds., pp. 351-372, W. H. Freeman, New York; Goding. 1986. Monoclonal Antibodies: Principles and Practice. Academic Press, San Diego). The fused cells are cloned and screened for production of the desired anti-peptide monoclonal antibody using immunological assays such as ELISAs. If desired, purification of monoclonal antibody from hybridoma culture supernatants or ascites fluid may be accomplished using methods known in the art, e.g., Protein G or peptide affinity column chromatography.

The isolated polyclonal and monoclonal antibodies produced in response to immunization with the peptides may be used in immunoassays for detection of P. vivax. The antibodies may be used intact or fragments may be generated which are also capable of binding to the peptide and to ESP-1 protein (Fab or F(ab′)₂). Intact antibodies as well as antigen binding fragments thereof are intended to be encompassed by the present invention. While immunoassays can be performed using only polyclonal antibody reagents, in most cases monoclonal antibody or a combination of polyclonal and monoclonal antibodies are preferred. In general, antibodies or antigens in immunoassays are labeled by conjugation to a detectable label to facilitate detection of antigen/antibody binding by inclusion of the label in the binding complex. As used herein, the term “label”, “detectable label” and related terms are intended to encompass both the detectable label alone and, as described below, detectable labels associated with particles. Suitable labels and methods for conjuating them to proteins such as antibodies are well known. Directly detectable labels, which do not require additional reagents or reaction to be detected, include radioisotopes, fluorescent dyes and visible asorbing dyes. Enzymes capable of reacting to produce colored products are suitable indirectly detectable labels commonly used for conjugation to antibodies in specific binding assays. All of the foregoing labels are suitable for conjugation to the polyclonal and monoclonal antibodies of the invention.

Particulate detectable labels are preferred for conjugation to the antibodies. Such particles include particles of polymers (e.g., latex or polystyrene), sacs, liposomes, metallic sols (e.g., colloidal silver or colloidal gold), other colloidal particles and polymeric dyes. To form the particulate label, the particles are derivatized to include the selected detectable label, usually by formation of a chemical bond using methods known in the art for this purpose. Polymer particles, such as latex particles, may also have the dye incorporated into the polymer. In the case of sacs and liposomes, the label may also be entrapped in the vesicle. The particle and its associated label may then be chemically conjugated to the antibody for use in specific binding assays. Alternatively, polymer particles, polymeric dyes and metal particles may be coated with the antibody as described in U.S. Pat. No. 5,096,837. The preferred detectable labels for association with the present antibodies are liposomes encapsulating an entrapped visible dye or other colored particles, with the antibody coupled to the surface of the liposome or particle. Such liposome labels are described in U.S. Pat. No. 4,695,554.

Protocols for immunoassays using the antibodies of the invention are well known in the art. For example, polyclonal or monoclonal antibodies according to the invention or antigen binding fragments thereof may be employed in sandwich assays for detection of PvESP-1 or in any of the known modifications and variations of sandwich assay protocols. Alternatively, the antibodies and antigen binding fragments thereof may be employed in various competitive assay formats as are known in the art. The basics of these assay protocols are reviewed in Current Protocols in Immunology, supra. When used as a diagnostic for P. vivax malarial infection, it is preferred that the sample tested for the presence of PvESP-1 protein be either lysed or unlysed blood. However, other samples may be assayed as well, for example, supernatants of infected cell cultures, extracts of P. vivax parasites, serum, plasma, urine and cerebrospinal fluid.

Devices for performing specific binding assays, especially immunoassays, are known and can be readily adapted for use with the present monoclonal and polyclonal antibodies for detection of PvEP-1 protein. Solid-phase assays, in general, are easier to perform than heterogeneous assay methods such as precipitation assays because separation of reagents is faster and simpler. Solid-phase assay devices include microtiter plates, flow-through assay devices, dipsticks and immunocapillary or immunochromatographic immunoassay devices as described in U.S. Pat. No. 4,743,560; U.S. Pat. No. 4,703,017; U.S. Pat. No. 4,666,866; U.S. Pat. No. 4,366,241; U.S. Pat. No. 4,818,677; U.S. Pat. No. 4,632,901; U.S. Pat. No. 4,727,019; U.S. Pat. No. 4,920,046; U.S. Pat. No. 4,855,240; U.S. Pat. No. 5,030,558, and; U.S. Pat. No. 4,168,146. Most preferred are immunocapillary assay devices which can be used as a dipstick, employing the inventive monoclonal and/or polyclonal antibodies.

In one embodiment of the PvESP-1 assay, an immunocapillary dipstick assay device is designed for conducting a sandwich immunoassay for PvESP-1 antigen. The device comprises a piece of microporous absorbent material such as nitrocellulose laminated to a plastic backing. In contact with the microporous material is a strip of a second absorbent material such as glass fiber, also laminated to the plastic backing. Nitrocellulose is preferred for the first material because it allows immobilization of protein simply by applying the protein solution to the nitrocellulose and allowing it to be absorbed. The second absorbent also absorbs the fluids which pass through the microporous material. An anti-PvESP-1 monoclonal antibody (e.g., mAb 1D11.G10 or, preferably a monoclonal antibody according to the invention) is immobilized on the microporous absorbent in a position where it will not be directly immersed in the sample being tested.

To perform the assay, the portion of the microporous absorbent below the monoclonal antibody is contacted with the sample such that the sample fluid is drawn up into it by capillarity (wicking), thus bringing the sample into contact with the antibody and allowing binding between the antibody and any PvESP-1 antigen which may be present in the sample. Thereafter, a solution containing a polyclonal anti-PvESP-1 antibody with a detectable label is wicked up into the microporous absorbent into contact with the monoclonal antibody/bound antigen complex such that the polyclonal antibody binds to the complexes through interaction with PvESP-1. Optionally, a wash solution containing a mild detergent may be wicked into the microporous absorbent after binding of the polyclonal antibody. The detectable dye label is then visualized in the area of the immobilized monoclonal antibody if PvESP-1 is present in the sample.

A positive control area may be included on the microporous absorbent in the vicinity of but distinct from the immobilized monoclonal antibody. The positive control may be PvESP-1 antigen, the immunogenic peptides, or a derivative or analog thereof which also binds the antibodies of the invention. In addition, a capture antibody for a second malarial antigen (e.g., HRP-II) may be included on the microporous absorbent to provide simultaneous detection and identification of the antigens of different Plasmodium species. To detect the two antigens, the detector antibody solution comprises a mixture of two labeled polyclonal antibodies, each of which is specific for one of the two Plasmodium species antigens.

The devices and reagents for performing the immunoassays of the invention may be packaged in the form of a kit for convenience. For example, such a kit may include an appropriate assay device, antibody reagents, reagents for development of the assay such as buffers and, if needed reagents for detection of the detector antibody label.

The inventive antibodies may also be useful for reducing the risk of P. vivax infection or treating such infection once established. Treatment may be accomplished by administering to an animal suffering from malaria infection, preferably a human, a therapeutically effective amount of a pharmaceutical composition comprising a monoclonal or polyclonal antibody according to the invention. In addition, pharmaceutically acceptable compositions comprising antibody or peptide may be administered to an animal in a dose sufficient to increase immunity to subsequent P. vivax infection. Alternatively, anti-idiotypic antibodies raised against the inventive monoclonal or polyclonal antibodies may also be administered in a pharmaceutical composition as a vaccine against malaria infection.

The following experimental Examples are intended to illustrate certain features and embodiments of the invention but are not to be considered as limiting the scope of the invention as defined by the appended claims.

### EXAMPLE 1

A 14-mer peptide having the amino acid sequence of SEQ ID NO:1 with an added N-terminal Cys-Gly was synthesized, deprotected and purified to greater than 95% purity using conventional techniques. The purified peptide was conjugated to sulfo-SMCC derivatized keyhole limpet hemocyanin (KLH) essentially as described by Rothbard, et al. (1984. J. Exp. Med. 160:208-221). Peptide conjugate was isolated from unconjugated peptide by chromatography on Sephadex™ G25 and used to immunize New Zealand white rabbits. The antigen was suspended in saline, emulsified by mixing with an equal volume of Freund's Adjuvant, and injected into three to four subcutaneous dorsal sites. Sera were collected prior to immunization and after three immunizations. Antibody titers were determined in ELISAs using the inventive peptide conjugated to an alternative carrier (e.g., BSA) immobilized on the solid phase. Preimmune sera were tested at the same time as production sera. Results were expressed as the reciprocal of the serum dilution that resulted in an OD₄₉₂ of 0.200 upon detection with HRP-anti-rabbit IgG conjugate and peroxidase dye. Prebleed titers for the two rabbits were less than 50. Following immunization the titers were 142,000 and 131,000.

Following Protein G purification the anti-14-mer antibodies from each rabbit were evaluated in Western blots for recognition of recombinant PvESP-1. mAb 1D11.G10 was included as a positive control. Antibody binding to PvESP-1 was detected using goat anti-mouse polyvalent immunoglobulins (Sigma) and goat anti-rabbit IgG (Cappel). The labels were developed with FAST™ BCIP/NBT (Sigma). The anti-14-mer polyclonal antibody produced by one rabbit reacted with intensity equal to Mab 1D11, while the antibody produced by the other rabbit reacted with slightly less intensity.

The anti-14-mer polyclonal antibodies were further tested in a solid phase dipstick immunoassay for detection of P. vivax. Immunocapillary dipstick devices were constructed by laminating nitrocellulose to adhesive plastic backing strips. The nitrocellulose was spotted with a monoclonal P. vivax capture antibody in a reaction area and with a recombinant PvESP-1 antigen in a positive control area. The nitrocellulose was also spotted with a monoclonal P. falciparum capture antibody in a separate reaction area and with a recombinant HRP-II antigen in a separate positive control area. The strips were blocked, dried and laminated to a wide adhesive strip with a strip of glass fiber wick overlapping the membrane strip. Whole blood or serum samples were added to the end of the membrane strip and wicked up into the nitrocellulose into contact with the immobilized antibodies. Following this, a solution comprising the anti-PvESP-1 polyclonal antibodies and anti-HRP-II polyclonal antibodies both coupled to liposomes containing an entrapped dye was added to the end of the membrane strip and wicked up into contact with the capture antibodies and any antigen bound thereto. A wash reagent containing a mild detergent was then wicked into the nitrocellulose.

In positive samples, the dye was visible at the site of the P. vivax monoclonal capture antibodies, indicating that the causative agent of the infection was P. vivax. No reaction was observed at the site of the P. falciparum monoclonal capture antibody, indicating that no P. falciparum infection was present. Detection of the dye at the site of the HRP-II and PvESP-1 antigen positive controls confirmed that the assay was working properly. The results are shown in the following Table:

| Patient# | Parasites/μl | Vivax Reaction | Falciparum Reaction |
|---|---|---|---|
| Monkey SI-846 | 240 | 1.5 | 0 |
| Monkey 113-94 | 6390 | 3 | 0 |
| Monkey SI-758 | 21,960 | 2 | 0 |
| M.M. | 154 | 1 | 0 |
| M.V. | 385 | 1 | 0 |
| P.L. | 455 | 0.5 | 0 |
| B.M. | 700 | 1 | 0 |
| R.V. | 1260 | 0.5 | 0 |

These results demonstrate that when used for detection of P. vivax the antibodies of the invention are capable of detecting parasitemias as low as 154 parasites/μl. This represents a significantly improves sensitivity as compared to the anti-PvESP-1 antibodies of the prior art.

What is claimed is:

1. An isolated antibody produced in response to immunization of a mammal with a peptide consisting of SEQ ID NO:1 and, optionally, an N-terminal Cys-Gly linker coupled to an immunogenic carrier.

2. The antibody of claim 1 which is a polyclonal antibody.

3. The antibody of claim 1 which is a monoclonal antibody.

4. A hybridoma which produces the monoclonal antibody of claim 3.

5. A peptide consisting of SEQ ID NO:1 and, optionally, an N-terminal Cys-Gly linker.

6. The peptide of claim 5 which is coupled to an immunogenic carrier.

7. The peptide of claim 6 wherein the immunogenic carrier is KLH.

8. A method for producing antibodies specific for Plasmodium vivax comprising:
a) immunizing a mammal with a peptide consisting of SEQ ID NO:1 and, optionally an N-terminal Cys-Gly linker coupled to an immunogenic carrier, and;
b) isolating antibodies produced in response to the peptide from the immunized mammal.

9. The method of claim 8 wherein the peptide is coupled to KLH.

10. The method of claim 8 wherein polyclonal antibodies are isolated.

11. The method of claim 8 wherein monoclonal antibodies are isolated.

12. A method for detecting Plasmodium vivax comprising contacting a sample suspected of containing a P. vivax antigen with the antibody of claim 1 and detecting binding of the antibody to the antigen as an indication of the presence of the antgen.

13. The method of claim 12 wherein binding is detected in a sandwich immunoassay.

14. The method of claim 13 wherein the antibody of claim 1 is a polyclonal antibody and the P. vivax antigen is captured by binding to an anti-PvESP-1 monoclonal antibody on a solid phase prior to detecting binding of the polyclonal antibody of claim 1 to the captured antigen.

15. The method of claim 12 wherein the antibody of claim 1 is a monoclonal antibody.

16. The method of claim 12 wherein binding is detected in a competitive immunoassay.

17. The method of claim 12 wherein binding is detected by means of a particulate detectable label conjugated to the antibody.

18. The method of claim 12 which is performed using an immunocapillary assay device.

19. A kit for performing an assay for a Plasmodium vivax antigen comprising:
a) a polyclonal antibody according to claim 1 coupled to a detectable label;
b) an anti-PvESP-1 monoclonal antibody immobilized on a solid phase of an immunocapillary immunoassay device, and;
c) reagents for performing an assay for the antigen using the immunocapillary immunoassay device.
